(19) United States
(12) Patent Application Publication
(10) Pub. No.: US 2020/0245874 A1
(43) Pub. Date: Aug. 6, 2020

(54) ECG SIGNAL PROCESSOR, PERSONAL IDENTIFICATION SYSTEM, AND ECG SIGNAL PROCESSING METHOD

(71) Applicant: Panasonic Intellectual Property Management Co., Ltd., Osaka (JP)

(72) Inventor: Akinori MATSUMOTO, Osaka (JP)

(21) Appl. No.: 16/633,029

(22) PCT Filed: May 29, 2018
(86) PCT No.: PCT/JP2018/020430

§ 371 Date: Jan. 22, 2020

(30) Foreign Application Priority Data
Jul. 25, 2017 (JP) 2017-144033

(51) Int. Cl. A61B 5/0245; A61B 5/021; A61B 5/00; A61B 5/053

(52) U.S. Cl. CPC A61B 5/0245; A61B 5/02116; A61B 5/6801; A61B 5/053; A61B 5/7285

(57) ABSTRACT

ECG signal processor includes: a signal processing circuit configured to amplify an ECG signal detected by an electrode attached to a living body and output the ECG signal amplified; and a common-mode signal generation circuit configured to generate a common-mode signal for increasing an amplitude of a peak of the ECG waveform indicated by the ECG signal using the ECG signal amplified by the signal processing circuit and apply the common-mode signal generated to the electrode.

### TECHNICAL FIELD

The present invention relates to an electrocardiogram (ECG) signal processor, a personal identification system, and an ECG signal processing method, and specifically to a technique improving accuracy in personal identification using ECG signals.

### BACKGROUND ART

ECG signals are electrical signals caused by periodic heart motion. It is known that each person has unique waveform pattern per period of the heart motion (hereinafter referred to as a “heartbeat pattern”). Personal identification techniques using ECG signals based on the fact are suggested (see, e.g., Patent Literature (PTL) 1).

According to PTL 1, a measurement system includes a bioimpedance measurement unit and an ECG signal measurement unit operating simultaneously in parallel. This allows personal identification using ECG signals after determining if there is any measurement error, for example, based on an obtained bioimpedance, that is, highly reliable personal identification.

### CITATION LIST

### Patent Literature

PTL 1: Japanese Unexamined Patent Application Publication No. 2012-210236

### SUMMARY OF THE INVENTION

### Technical Problem

In the technique of PTL 1, assume that what is called a “dry electrode” formed without using any conductive paste is employed for measurement. At a high contact impedance between the electrode and a living body, no stable ECG signals are obtained, which hinders highly accurate personal identification. This is because ECG signals are affected by disturbance noise such as mains hum at a high contact impedance, which causes unstable peaks of the P, Q, R, S, T, and U waves of heartbeat patterns.

The present invention was made to solve the problem. It is an objective of the present invention to, for example, an ECG signal processor capable of stably measuring ECG signals even at a high contact impedance between an electrode and a living body.

### Solutions to Problem

In order to achieve the objective, an ECG signal processor according to an aspect of the present invention includes: a signal processing circuit configured to amplify an ECG signal detected by an electrode attached to a living body and output the ECG signal amplified; and a common-mode signal generation circuit configured to generate a common-mode signal for increasing an amplitude of a peak of an ECG waveform indicated by the ECG signal using the ECG signal amplified by the signal processing circuit and apply the common-mode signal generated to the electrode.

In order to achieve the objective, a personal identification system according to an aspect of the present invention includes: the ECG signal processor described above; a storage unit configured to store, as register information, features of ECG waveforms indicated by ECG signals output from the signal processing circuit included in the ECG signal processor in association with a plurality of users; and an identification unit configured to compare a feature of the ECG waveform of a subject indicated by the ECG signal output from the signal processing circuit included in the ECG signal processor with the register information stored in the storage unit to identify the subject among the plurality of users.

In order to achieve the objective, an ECG signal processing method according to an aspect of the present invention includes: An electrocardiogram (ECG) signal processing method, comprising: obtaining an ECG signal detected by an electrode attached to a living body; and generating a common-mode signal for increasing an amplitude of a peak of an ECG waveform indicated by the ECG signal obtained in the obtaining of the ECG signal and applying the common-mode signal generated to the electrode.

### Advantageous Effect of Invention

The present invention achieves an ECG signal processor and an ECG processing method capable of stably measuring ECG signals even at a high contact impedance between an electrode and a living body, and a personal identification system including the ECG signal processor.

### BRIEF DESCRIPTION OF DRAWINGS

FIG. 1 is an external view illustrating a configuration of a personal identification system according to an embodiment.

FIG. 2A illustrates example arrangement of electrodes included in an ECG signal processor shown in FIG. 1.

FIG. 2B illustrates that a subject is seated on the ECG signal processor shown in FIG. 2A.

FIG. 3 illustrates an ECG signal processor according to another embodiment.

FIG. 4 illustrates an ECG signal processor according to further another embodiment.

FIG. 5 illustrates example shapes of the electrodes included in the ECG signal processor.

FIG. 6 is a block diagram illustrating a configuration of a personal identification system according to an embodiment.

FIG. 7 is a block diagram illustrating a specific configuration of an ECG signal processor shown in FIG. 6.

FIG. 8 illustrates a heartbeat pattern obtained by an ECG signal.

FIG. 9 is a flow chart showing processing of the ECG signal processor of the personal identification system according to the embodiment.

FIG. 10 is a flow chart showing processing of an information processor of the personal identification system according to the embodiment.

FIG. 11 illustrates example display of a display unit while the information processor performs personal identification.

FIG. 12 illustrates features of the heartbeat pattern of the ECG waveform.

FIG. 13 illustrates an example waveform indicated by an ECG signal (hereinafter referred to as “registered data A”) on which no common-mode signal is superimposed by the ECG signal processor.

FIG. 14 illustrates an example waveform indicated by an ECG signal (hereinafter referred to as “registered data B”) on which a common-mode signal is superimposed by the ECG signal processor.

FIG. 15 illustrates another example waveform indicated by another ECG signal (hereinafter referred to as “registered data C”) on which no common-mode signal is superimposed by the ECG signal processor.

FIG. 16 illustrates results of personal identification using the ECG waveforms of registered data A to C after registering the features of the ECG waveforms.

FIG. 17 is a block diagram illustrating a configuration of an ECG signal processor according to a variation of the embodiment.

FIG. 18 illustrates an example waveform indicated by an ECG signal on which a common-mode signal is superimposed by the ECG signal processor according to the variation.

### DESCRIPTION OF EXEMPLARY EMBODIMENTS

Now, embodiments of the present invention will be described in detail with reference to the drawings. Note that the embodiments described below are mere preferred specific examples of the present invention. The numerical values, shapes, materials, constituent elements, the arrangement and connection of the constituent elements, steps, step orders etc. shown in the following embodiments are thus mere examples, and are not intended to limit the scope of the present invention. Among the constituent elements in the following embodiments, those not recited in any of the independent claims defining the broadest concept of the present invention are described as optional constituent elements. The figures are not necessarily drawn strictly to scale. In the figures, substantially the same constituent elements are assigned with the same reference marks, and redundant descriptions will be omitted or simplified.

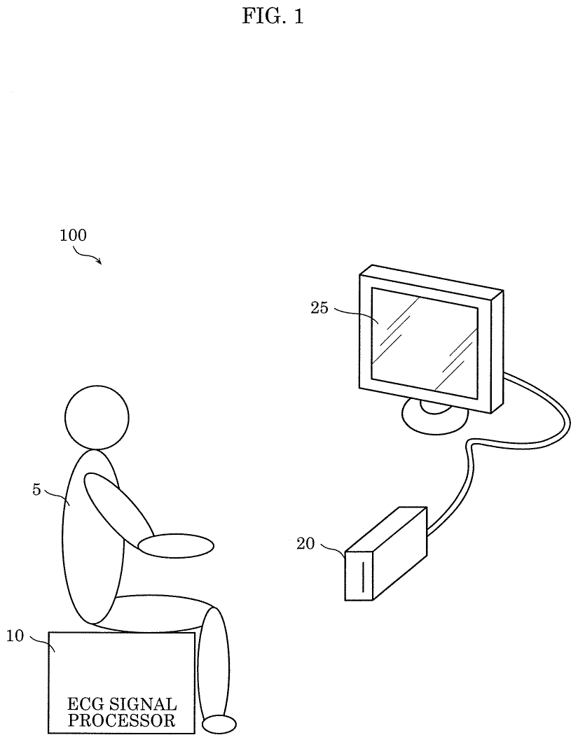

FIG. 1 is an external view illustrating a configuration of personal identification system 100 according to an embodiment. This figure also includes subject 5 to be subjected to personal identification.

Personal identification system 100 performs personal identification of subject 5, and includes ECG signal processor 10, information processor 20, and display unit 25.

ECG signal processor 10 is a measurement device configured as a chair on which subject 5 is seated. The device measures ECG signals on the backs of thighs (i.e., hamstrings) of subject 5 and sends the measured ECG signals wireless to information processor 20. Note that ECG signal processor 10 does not necessarily have a chair structure. ECG signal processor 10 may be attached to a chair structure as a separate body.

Information processor 20 performs personal identification of subject 5 using the ECG signals sent wireless from ECG signal processor 10 and causes display unit 25 to display the result. Note that information processor 20 may be, for example, a computer device including, for example, a non-volatile memory, such as a hard disk or a ROM, storing programs; a RAM temporarily storing information; a processor executing programs; or input/output ports to be connected to peripherals. Information processor 20 may be, for example, a portable information terminal such as a personal computer or a smartphone.

Display unit 25 is a display such as liquid crystal display (LCD) that displays, for example, results of personal identification performed by information processor 20. Note that an output device constituting personal identification system 100 may be a sound output device in place of display unit 25 or in addition to display unit 25.

Note that this personal identification system 100 may include an input device (not shown), such as a remote controller or buttons, through which subject 5 gives instructions to ECG signal processor 10 and information processor 20. The input device may be an independent device connected wired or wireless to ECG signal processor 10 and information processor 20, or a device incorporated and fixed into ECG signal processor 10 or information processor 20.

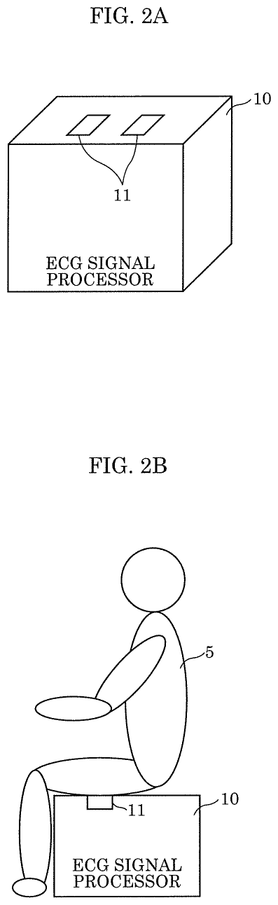

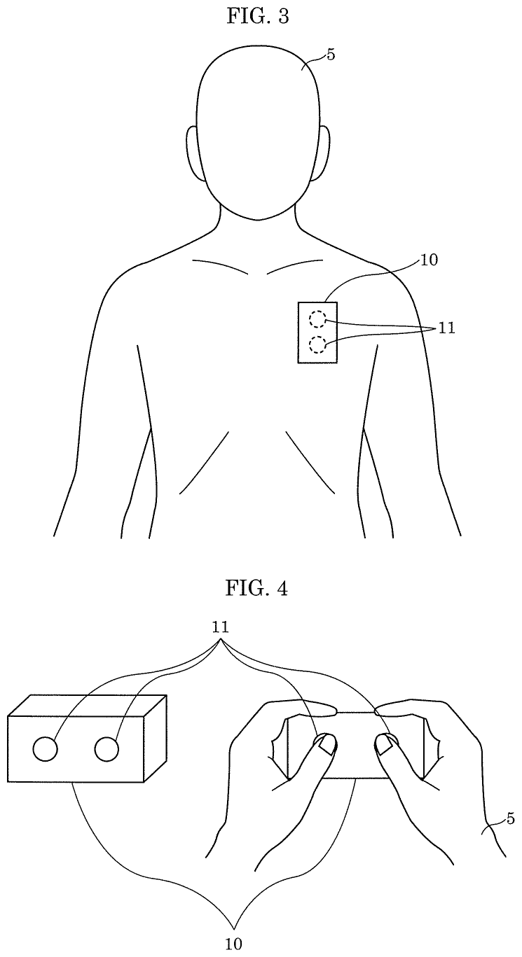

FIG. 2A illustrates example arrangement of electrodes 11 included in ECG signal processor 10 shown in FIG. 1. In this embodiment, electrodes 11 are located in two positions (as a measurement electrode and a reference electrode) on the upper surface of the chair structure in the shape of a rectangular prism. When subject 5 is seated on ECG signal processor 10 with the rectangular prism chair structure, the backs of the thighs of subject 5 touch the respective electrodes. Electrodes 11 may be made of, for example, gold, silver, or silver/silver chloride (Ag/AgCl). Note that electrodes 11 are not necessarily included in ECG signal processor 10 and may be attached to subject 5 in advance.

FIG. 2B illustrates that subject 5 is seated on ECG signal processor 10 shown in FIG. 2A. Electrodes 11 are located on the backs of the thighs of subject 5. Note that subject 5 does not necessarily expose the thighs and may wear clothes such as pants. Through the clothes, ECG signals on the backs of the thighs of subject 5 are detected by electrodes 11. This is because ECG signal processor 10 stably measures ECG signals in this embodiment even at a high contact impedance between the electrodes and the living body.

Note that the states and arrangements of ECG signal processor 10 and electrodes 11 are not limited to those shown in FIGS. 1, 2A, and 2B and may be those shown in FIGS. 3 and 4, for example.

FIG. 3 illustrates ECG signal processor 10 according to another embodiment. In this embodiment, ECG signal processor 10 is a patch-type ECG sensor attached to the left chest of subject 5 with electrodes 11 interposed therebetween.

FIG. 4 illustrates ECG signal processor 10 according to further another embodiment. In this embodiment, ECG signal processor 10 may have a structure like a small portable controller including electrodes 11 to be touched by the thumbs of subject 5, in two parts of the front surface of a case in a shape of rectangular prism.

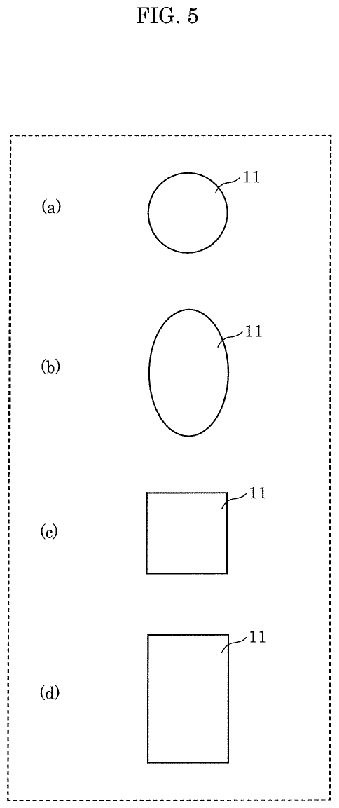

FIG. 5 illustrates example shapes of electrodes 11 included in ECG signal processor 10. Electrodes 11 may be in the shape of a circle shown in (a) of FIG. 5, an oval shown in (b) of FIG. 5, a square shown in (c) of FIG. 5, a rectangular shown in (d) of FIG. 5, or may be a combination of these shapes (a combination of electrodes n).

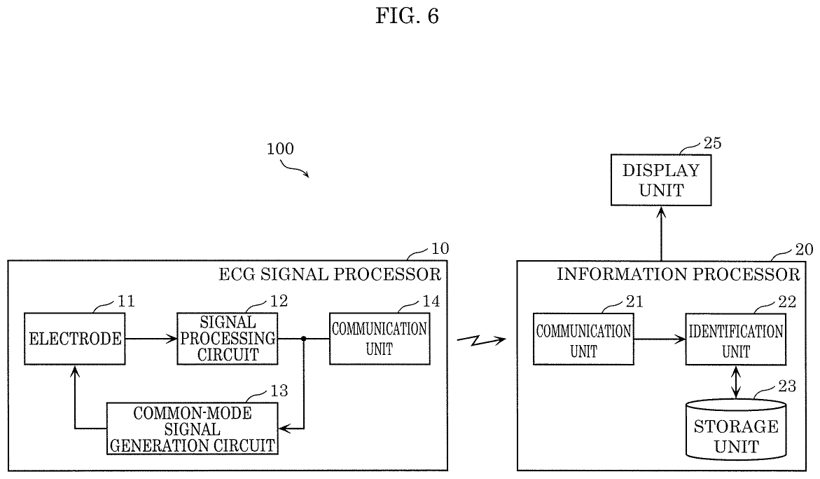

FIG. 6 is a block diagram illustrating a configuration of personal identification system 100 according to this embodiment. Personal identification system 100 includes ECG signal processor 10, information processor 20, and display unit 25.

ECG signal processor 10 includes electrodes 11, signal processing circuit 12, common-mode signal generation circuit 13, and communication unit 14.

As shown in FIGS. 2A and 2B, electrodes 11 are electrodes (including a measurement electrode and a reference electrode) attached to a living body and may be not only dry electrodes but also wet electrodes. Note that “attached to a living body” means that the electrodes are located near on the living body to allow measurement of ECG signals from the living body. This includes not only the electrodes in direct contact with the skin of the living body but also the electrodes fixed relative to the living body with clothes interposed therebetween.

Signal processing circuit 12 amplifies the ECG signals detected by electrodes 11 that are attached to the living body and outputs an amplified signal.

Common-mode signal generation circuit 13 uses the ECG signal amplified by signal processing circuit 12 to generate a common-mode signal for increasing the peak amplitude of an ECG waveform indicated by the ECG signal. The circuit applies then the generated common-mode signal to one of electrodes 11.

Communication unit 14 is a communication interface that sends information on the ECG signals output from signal processing circuit 12 to information processor 20. The communication unit may be, for example, a wireless communication adapter for Bluetooth (registered trademark) or Wi-Fi (registered trademark). The “information on the ECG signals” meant here includes at least one of the ECG signals and the features obtained by signal processing of the ECG signals (e.g., information on the peaks of the ECG waveform). Note that communication unit 14 is not limited to the interface for wireless communications but may be an interface for wired communications.

Although not shown in the figure, ECG signal processor 10 includes a power supply circuit that supplies direct current (DC) power to signal processing circuit 12, common-mode signal generation circuit 13, and communication unit 14. The power supply circuit may be a battery, a DC/DC converter that converts the voltage of a battery to a required DC voltage, or a regulator circuit that generates a constant DC voltage from a commercial power supply.

Information processor 20 includes communication unit 21, identification unit 22, and storage unit 23.

Communication unit 21 is a communication interface that receives the information on the ECG signals sent from ECG signal processor 10. The communication unit may be, for example, a wireless communication adapter for Bluetooth (registered trademark) or Wi-Fi (registered trademark). Note that communication unit 21 is not limited to the interface for wireless communications but may be an interface for wired communications.

Storage unit 23 is a device that stores, as register information, the features of ECG waveforms indicated by the ECG signals output from signal processing circuit 12 included in ECG signal processor 10 in association with a plurality of users (i.e., user identifiers). The storage unit may be, for example, a hard disk.

Identification unit 22 is a processing unit that compares the features of the ECG waveform indicated by the ECG signals of subject 5 output from signal processing circuit 12 of ECG signal processor 10, to the register information stored in storage unit 23. The dentification unit identifies then the subject among the plurality of users. Identification unit 22 causes display unit 25 to display the identification result. Such identification unit 22 is achieved by the programs executed by the processor included in information processor 20 as described above. Note that identification unit 22 performs not only such personal identification but also processing of obtaining information to be registered and causes storage unit 23 to register the information. Specifically, identification unit 22 extracts the features required for personal identification from the ECG signals sent from ECG signal processor 10 or obtains the features sent from ECG signal processor 10. The identification unit causes storage unit 23 to store, as register information, the extracted or obtained features in association with subject 5.

Although not shown in the figure, information processor 20 includes a power supply circuit that supplies DC power to communication unit 21, identification unit 22, and storage unit 23. The power supply circuit may be, for example, a regulator circuit that generates a constant DC voltage from a commercial power supply.

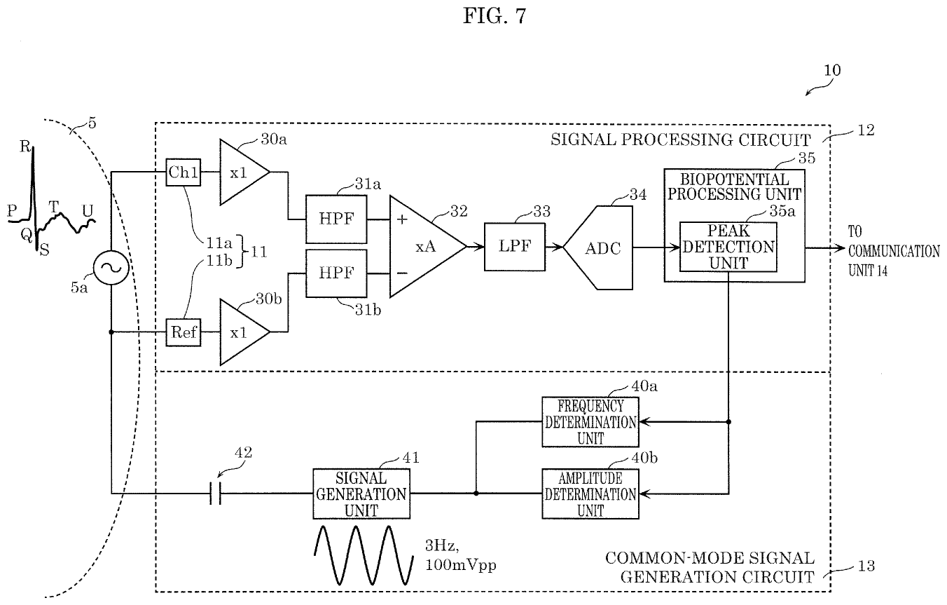

FIG. 7 is a block diagram illustrating a specific configuration of ECG signal processor 10 shown in FIG. 6. This figure shows specific circuit diagrams of signal processing circuit 12 and common-mode signal generation circuit 13 constituting ECG signal processor 10. Note that the left of the figure also shows an equivalent circuit (i.e., signal source 5a of the ECG signals) of subject 5.

Signal processing circuit 12 includes electrodes 11 (i.e., measurement electrode 11a and reference electrode 11b), buffer amplifiers 30a and 30b, high-pass filters 31a and 31b, differential amplifier 32, low-pass filter 33, A/D converter 34, and biopotential processing unit 35.

Measurement electrode 11a and reference electrode 11b are an electrode for measuring ECG signals and an electrode for measuring a reference voltage, respectively.

Buffer amplifiers 30a and 30b are circuits that perform impedance conversion with respect to the signals (i.e., potentials) detected by measurement electrode 11a and reference electrode 11b, respectively, and may be, for example, voltage followers. That is, each of buffer amplifiers 30a and 30b has a high input impedance and a low output impedance, and does not amplify voltages (i.e., has a voltage amplification factor of 1). In this specification, the term “amplifier(s)” includes not only those with a voltage amplification factor larger than 1, but also those performing only impedance conversion (with a voltage amplification factor of 1). Note that measurement electrode 11a and buffer amplifier 30a are integrated to form an active electrode. This also applies to reference electrode 11b and buffer amplifier 30b. Buffer amplifiers 30a and 30b may have a voltage amplification factor larger than 1.

High-pass filters 31a and 31b remove unnecessary low frequency components from the signals output from buffer amplifiers 30a and 30b, respectively. Each filter may be, for example, a CR filter or an active filter using an operational amplifier.

Differential amplifier 32 subtracts the signal output from high-pass filter 31b from the signal output from high-pass filter 31a and amplifies the obtained difference, and may be, for example, an operational amplifier. This differential amplifier 32 is an example of the circuit that amplifies the difference between the signal detected by measurement electrode 11a and the signal detected by reference electrode 11b. That is, each signal output from differential amplifier 32 is the ECG signal indicating the potential at measurement electrode 11a using the potential at reference electrode 11b as a reference.

Low-pass filter 33 removes an unnecessary high frequency component from each signal output from differential amplifier 32, and may be, for example, an active filter using a CR filter or an operational amplifier.

A/D converter 34 samples each signal output from low-pass filter 33 and converts the sampled signal into a digital signal, for example, performs sampling at 1 kHz and converts the signal into a 12-bit digital signal. This A/D converter 34 is an example of the A/D converter that converts each signal output from differential amplifier 32 into a digital signal.

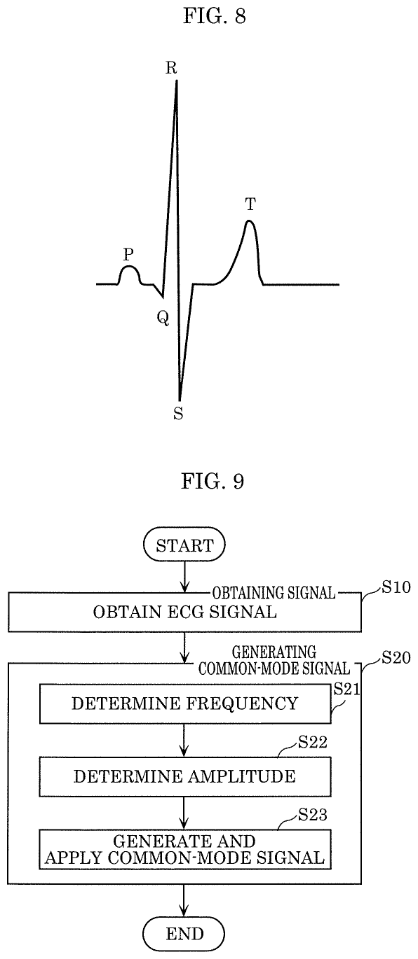

Biopotential processing unit 35 includes peak detection unit 35a that detects the peaks of the P wave, the Q wave, the R wave, the S wave, and the T wave of a heartbeat pattern from each signal (i.e., each digital ECG signal) output from A/D converter 34. The heartbeat pattern is as shown in FIG. 8. Specifically, peak detection unit 35a generates information on the peaks of the P wave, the Q wave, the R wave, the S wave, and the T wave (i.e., the signal indicating the timing and amplitude of the peaks) of the heartbeat pattern included in each ECG signal output from A/D converter 34. The processing unit outputs then the generated information on the peaks to frequency determination unit 40a and amplitude determination unit 40b of common-mode signal generation circuit 13.

Note that biopotential processing unit 35 basically sends the signals (i.e., each digital ECG signal) output from A/D converter 34 unchanged via communication unit 14 to information processor 20. Depending on the advance settings (e.g., instructions through the input device (not shown)), biopotential processing unit 35 sends, as the features, information on the peaks detected by peak detection unit 35a in addition to the ECG signals via communication unit 14 to information processor 20.

While biopotential processing unit 35 is included in ECG signal processor 10 in this embodiment, the configuration is not limited thereto. Instead or in addition, biopotential processing unit 35 may be included in information processor 20. In this case, the signals output from AM converter 34 is sent via communication unit 14 to information processor 20 to generate information on the peaks at peak detection unit 35a of biopotential processing unit 35 included in information processor 20. The generated information on the peaks is transmitted via communication unit 21 of information processor 20 and communication unit 14 of ECG signal processor 10 to ECG signal processor 10 to be used by frequency determination unit 40a and amplitude determination unit 40b.

Common-mode signal generation circuit 13 includes frequency determination unit 40a, amplitude determination unit 40b, signal generation unit 41, and coupling capacitor 42.

In a first mode, frequency determination unit 40a determines the frequency corresponding to the time lag between the peak of the P wave and the peak of the R wave of the ECG waveform. In a second mode, the frequency determination unit determines the frequency corresponding to the time lag between the peak of the Q or S wave and the peak of the T wave of the ECG waveform. Specifically, in the first mode, frequency determination unit 40a calculates the time lag between the peak of the P wave and the peak of the R wave using the information on the peaks detected by peak detection unit 35a. The frequency determination unit determines then the frequency with the calculated time lag as a period. In the second mode, frequency determination unit 40a calculates the time lag between the peak of the Q or S wave (e.g., the peak with a larger amplitude) and the peak of the T wave using the information on the peaks detected by peak detection unit 35a. The frequency determination unit determines then the frequency with the calculated time lag as a period. Note that the first mode or the second mode is selected based on the advance settings (e.g., instructions through the input device (not shown)).

Amplitude determination unit 40b determines the amplitude of the common-mode signal to be generated, based on the amplitude of the peaks of the ECG waveform. Specifically, amplitude determination unit 40b calculates the amplitude of the peak of the R wave (e.g., an average peak value of the R wave), which has the maximum amplitude among the peaks, using the information on the peaks detected by peak detection unit 35a. The smaller the obtained amplitude of the peak of the R wave is, the greater values are determined as the amplitude of the common-mode signal. For example, amplitude determination unit 40b stores, in advance, a table including a plurality of amplitude sections of the amplitude of the peak of the R wave in association with the amplitude of the common-mode signal to be determined. Amplitude determination unit 40b refers to the table to determine the amplitude of the common-mode signal corresponding to the amplitude of the peak of the R wave of the ECG waveform.

Signal generation unit 41 generates, as a common-mode signal, the signal with the frequency determined by frequency determination unit 40a and the amplitude determined by amplitude determination unit 40b. Specifically, signal generation unit 41 generates a sample data column with the frequency determined by frequency determination unit 40a and the amplitude determined by amplitude determination unit 40b. A built-in D/A converter converts the generated sample data column into an analog signal and causes the analog signal to pass through a built-in low-pass filter. Accordingly, the signal generation unit generates, as a common-mode signal for increasing the amplitude of the peaks of the ECG waveform, a sine wave signal with the frequency determined by frequency determination unit 40a and the amplitude determined by amplitude determination unit 40b (e.g., a sine wave signal with 3 Hz and 100 mVpp). Note that there is no need to synchronize the common-mode signal and the ECG waveform (i.e., to superimpose the peak of the sine wave of the common-mode signal and the peaks of the ECG waveform).

Coupling capacitor 42 is connected between an output terminal of signal generation unit 41 and reference electrode 11b, and allows only an AC component of the signal output from signal generation unit 41 to pass to be applied to reference electrode 11b. Coupling capacitor 42 may be, for example, a capacitor with 100 pF.

Note that digital signal processing in biopotential processing unit 35, frequency determination unit 40a, amplitude determination unit 40b, and signal generation unit 41 may be implemented by hardware using an exclusive logic circuit or by software using programs. As software, the digital signal processing may be implemented by a microcomputer including, for example, a non-volatile memory, such as a ROM, storing programs; a RAM temporarily storing information; processor executing programs; or input/output ports to be connected to peripherals.

Next, an operation of personal identification system 100 according to this embodiment configured as described above will be described.

FIG. 9 is a flow chart showing processing (i.e., an ECG signal processing method) of ECG signal processor 10 of personal identification system 100 according to this embodiment.

Signal processing circuit 12 obtain ECG signals detected by electrodes 11 (i.e., measurement electrode 11a and reference electrode 11b) attached to a living body (S10 of obtaining signals).

Specifically, the signal detected by measurement electrode 11a is subjected to impedance conversion at buffer amplifier 30a and an unnecessary low frequency component is removed by high-pass filter 31a. The signal is then input to a positive input terminal of differential amplifier 32. On the other hand, the signal detected by reference electrode 11b is subjected to impedance conversion at buffer amplifier 30b and an unnecessary low frequency component is removed by high-pass filter 31b. The signal is then input to a negative input terminal of differential amplifier 32. Differential amplifier 32 amplifies the difference between the signal input to the positive input terminal and the signal input to the negative input terminal. From the amplified signal, an unnecessary high frequency component is removed by low-pass filter 33. The signal is then converted into a digital ECG signal by A/D converter 34 to be input to biopotential processing unit 35. Biopotential processing unit 35 generates information on the peaks of the P wave, the Q wave, the R wave, the S wave, and the T wave of the heartbeat pattern included in the ECG signal output from A/D converter 34 (i.e., the signal indicating timing and amplitude of the peaks). The generated information is then output to common-mode signal generation circuit 13 (i.e., frequency determination unit 40a and amplitude determination unit 40b).

Next, a common-mode signal for increasing the amplitude of the peaks of the ECG waveform indicated by the ECG signals is generated in S10 of obtaining signals. The generated common-mode signal is applied to reference electrode 11b (S20 of generating a common-mode signal).

More specifically, frequency determination unit 40a determines the frequencies as follows (S21). In the first mode, the frequency determination unit determines the frequency corresponding to the time lag between the peak of the P wave and the peak of the R wave of the ECG waveform. In the second mode, the frequency determination unit determines the frequency corresponding to the time lag between the peak of the Q or S wave and the peak of the T wave of the ECG waveform. Specifically, in the first mode, frequency determination unit 40a calculates the time lag between the peak of the P wave and the peak of the R wave using the information on the peaks detected by peak detection unit 35a. The frequency determination unit determines then the frequency with the calculated time lag as a period. In the second mode, frequency determination unit 40a calculates the time lag between the peak of the Q or S wave (e.g., the peak with a larger amplitude) and the peak of the T wave using the information on the peaks detected by peak detection unit 35a. The frequency determination unit determines the frequency with the calculated time lag as a period.

Subsequently, amplitude determination unit 40b determines the amplitude of the common-mode signal to be generated based on the amplitude of the peaks of the ECG waveform (S22). Specifically, amplitude determination unit 40b calculates the amplitude of the peak of the R wave using the information on the peaks detected by peak detection unit 35a. The smaller the obtained amplitude of the peak of the R wave is, the greater value is determined as the amplitude of the common-mode signal.

Finally, signal generation unit 41 generates, as the common-mode signal, a signal with the frequency determined by frequency determination unit 40a and the amplitude determined by amplitude determination unit 40b. The signal generation unit applies then the generated signal through coupling capacitor 42 to reference electrode 11b (S23).

Note that S10 of obtaining signals and S20 of generating a common-mode signal are repeated in a certain period and performed simultaneously in parallel. Thus, once a common-mode signal is generated in S20 of generating a common-mode signal and applied to reference electrode 11b, the subsequent ECG signals are obtained in S10 of obtaining signals with the respective common-mode signals applied to reference electrode 11b, that is, with the common-mode signals superimposed on the ECG signals.

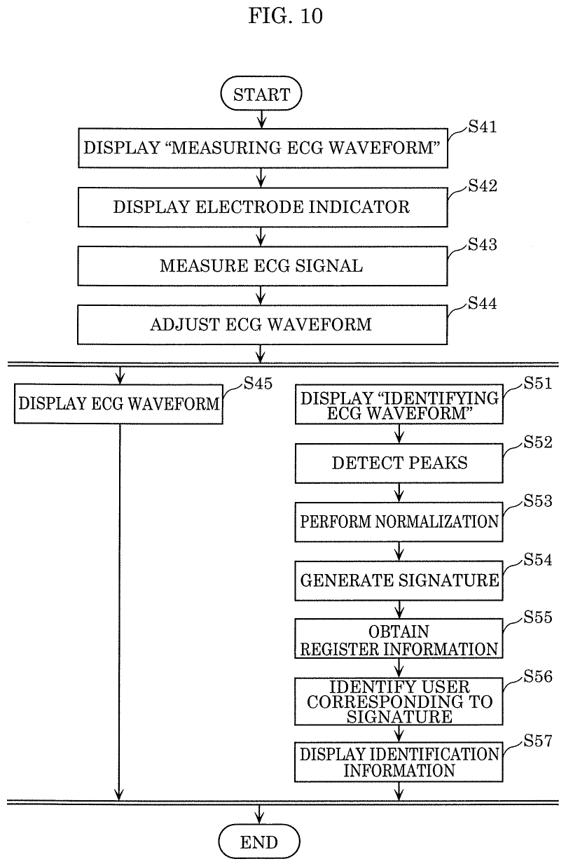

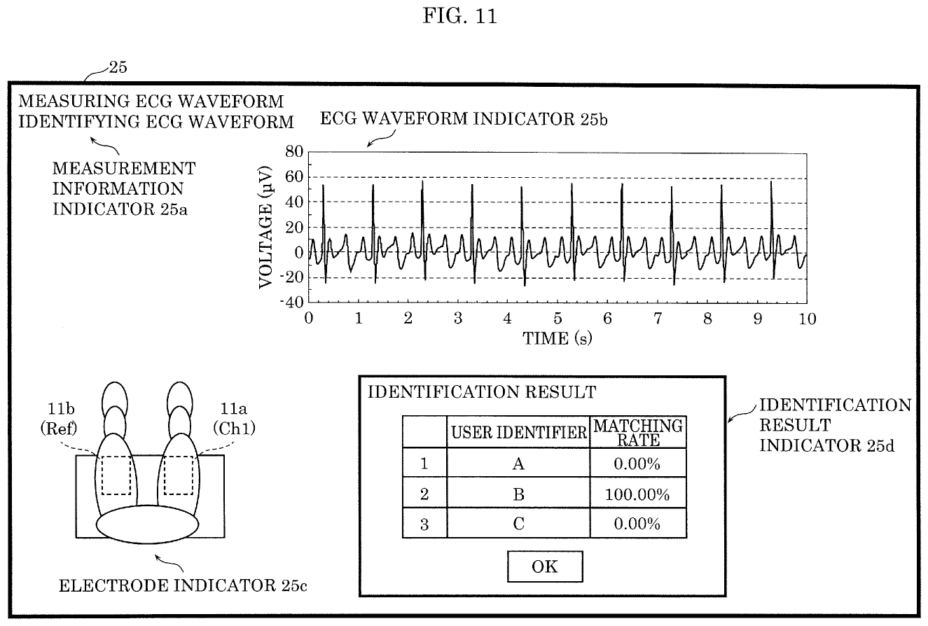

FIG. 10 is a flow chart showing processing (i.e., a personal identification method) of information processor 20 of personal identification system 100 according to this embodiment. FIG. 11 illustrates example display of display unit 25, when information processor 20 performs personal identification.

Once personal identification starts, identification unit 22 first causes measurement information indicator 25a of display unit 25 to indicate “measuring ECG waveform” (S41), and then to causes display electrode indicator 25c of display unit 25 to indicate the positions of the electrodes (S42).

Next, identification unit 22 instructs ECG signal processor 10 via communication unit 21 to cause ECG signal processor 10 to start measuring the ECG signals, and obtains the ECG signals via communication unit 21 of ECG signal processor 10 (S43). In order to extract information significant as an ECG waveform from the obtained ECG signals, identification unit 22 extracts a specific frequency component and calculates the power spectral density of the extracted frequency component to adjust the ECG waveform (S44).

After that, identification unit 22 causes ECG waveform indicator 25b of display unit 25 to indicate the adjusted ECG waveform (S45) and performs personal identification in parallel to the indication (S51 to S57).

During the personal identification (S51 to S57), identification unit 22 causes first measurement information indicator 25a of display unit 25 to display “identifying ECG waveform” (S51). Identification unit 22 performs, for example, differentiation of the adjusted ECG waveform to detect the peaks of the heartbeat pattern (S52), and calculates relative peak values of the peaks to normalize the amplitude of the ECG waveform (S53).

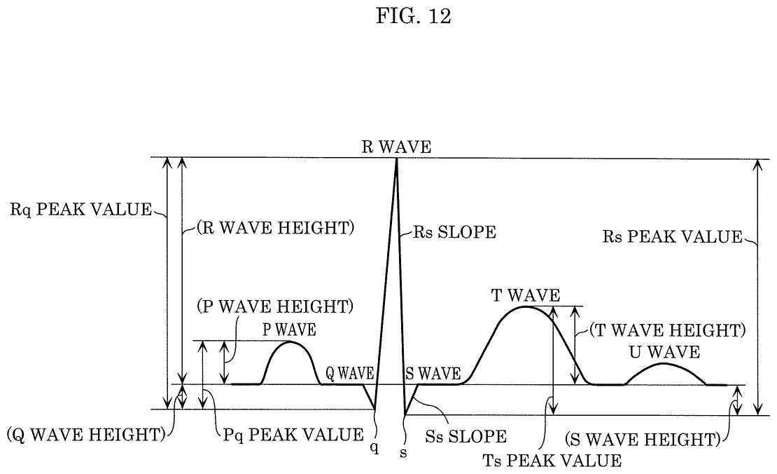

Subsequently, identification unit 22 generates, as a signature, the features of the heartbeat pattern shown in FIG. 12 from the normalized ECG waveform (S54). FIG. 12 illustrates the following as features. A “P wave height” indicates the height of the P wave. A “Q wave height” indicates the height of the Q wave. An “R wave height” indicates the height of the R wave. An “S wave height” indicates the height of the S wave. A “T wave height” indicates the height of the T wave. An “Rq peak value” indicates the difference in the height between the R wave and the Q wave. “Pq peak value” indicates the difference in the height between the P wave and the Q wave. A “Ts peak value” indicates the difference in the height between the T wave and the S wave. A “Rs peak value” indicates the difference in the height between the R wave and the S wave. An “Rs slope” indicates the slope extending from the R wave to the S wave. An “Ss slope” indicates the slope at the last of the peak of the S wave.

Next, identification unit 22 obtains register information stored in storage unit 23 (S55) and refers to the obtained register information to identify the user corresponding to the signature generated in S54 (S56). That is, the identification unit identifies, among the features included in the register information, features most resembling the signature, and outputs the information on the user (i.e., the user identifier) corresponding to the identified features as a result of personal identification.

Finally, identification unit 22 causes identification result indicator 25d of display unit 25 to indicate the result of personal identification (S57). Example display of identification result indicator 25d in FIG. 11 shows the results (percentages) of personal identification with respect to three user identifiers. Note that the three user identifiers are, for example, first to third most resembling user identifiers to a signature or user identifiers registered in advance.

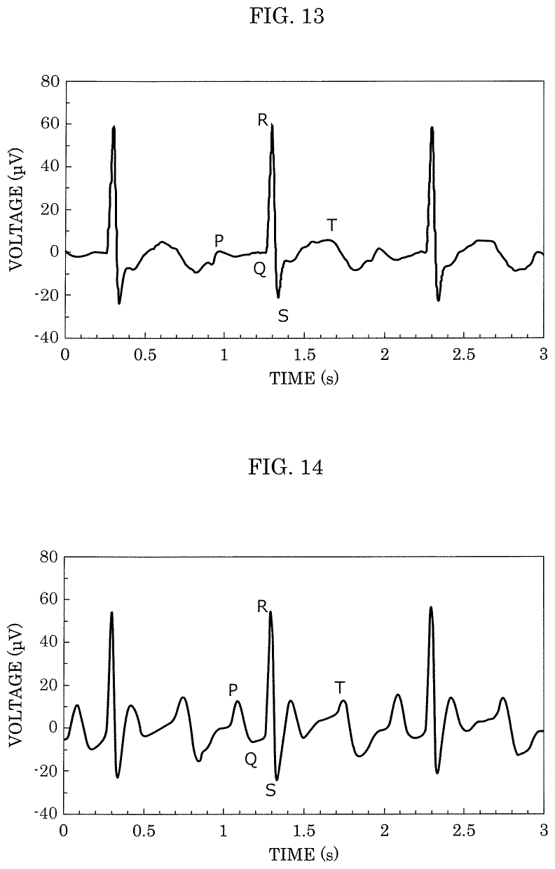

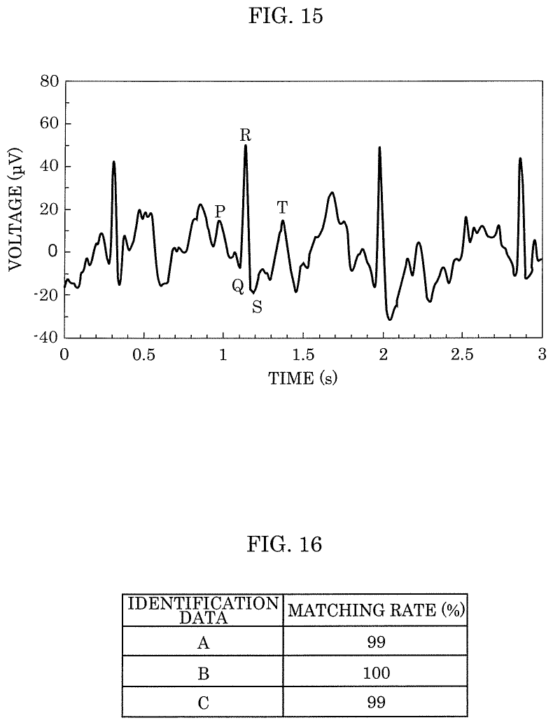

FIGS. 13 to 16 illustrate features of personal identification system 100 according to this embodiment. More specifically, FIG. 13 illustrates an example waveform (i.e., an original waveform) of an ECG signal (registered data A) on which no common-mode signal is superimposed by ECG signal processor 10. FIG. 14 illustrates an example waveform (i.e., a waveform for registration and identification) of an ECG signal (registered data B) on which a common-mode signal is superimposed by ECG signal processor 10. FIG. 15 illustrates an example waveform (i.e., a waveform for registration and identification) of another ECG signal (registered data C) on which no common-mode signal is superimposed by ECG signal processor 10. FIG. 16 illustrates results (percentages) of personal identification performed by identification unit 22 using waveforms of registered data A to C after registering in storage unit 23, the features of the ECG waveforms as register information.

As can be seen from FIG. 16, ECG signal processor 10 exhibits the highest matching rate (100%) when registering an ECG waveform using an ECG signal (registered data B) on which a common-mode signal is superimposed to perform personal identification. This may be because superimposition of the common-mode signal on the ECG signal increased the frequency of largely emphasizing the amplitude of the peaks of the ECG waveform and clarified the features of the ECG waveform.

As described above, ECG signal processor 10 according to this embodiment includes signal processing circuit 12 and common-mode signal generation circuit 13. Signal processing circuit 12 amplifies the ECG signals detected by electrodes 11 that are attached to a living body and outputs the signal after the amplification. Common-mode signal generation circuit 13 uses using the ECG signal amplified by signal processing circuit 12 to generate a common-mode signal for increasing the amplitude of the peaks of the ECG waveform indicated by the ECG signal. The common-mode signal generation circuit applies the generated common-mode signal to one of electrodes 11.

This configuration allows application of the common-mode signal for increasing the amplitude of the peaks of the ECG waveform indicated by the ECG signal to one of electrodes 11 and emphasis of the peaks of the heartbeat pattern indicated by the ECG signal. Accordingly, stable personal identification is possible even in the presence of disturbance noise. That is, an ECG signal processor capable of stably measuring ECG signals is provided even at a high contact impedance between electrodes 11 and the living body.

Common-mode signal generation circuit 13 includes frequency determination unit 40a and signal generation unit 41. Frequency determination unit 40a determines the frequency corresponding to the time lag between the peak of the P wave and the peak of the R wave of the ECG waveform. Signal generation unit 41 generates, as a common-mode signal, a signal with the frequency determined by frequency determination unit 40a.

This configuration allows application of the common-mode signal with the frequency corresponding to the time lag between the peak of the P wave and the peak of the R wave of the ECG waveform to one of electrodes 11. This increases the amplitude of the peaks of the P wave and the R wave of the heartbeat pattern that indicates the features of the subject. Accordingly, the processing of personal identification using the peaks of the P wave and the R wave of the heartbeat pattern stabilizes and the accuracy improves.

Alternatively, common-mode signal generation circuit 13 may include frequency determination unit 40a and signal generation unit 41. Frequency determination unit 40a determines the frequency corresponding to the time lag between the peak of the Q or S wave and the peak of the T wave of the ECG waveform. Signal generation unit 41 generates, as a common-mode signal, a signal with the frequency determined by frequency determination unit 40a.

This allows application of a common-mode signal with the frequency corresponding to the time lag between the peak of the Q or S wave and the peak of the T wave of the ECG waveform to one of electrodes 11. This increases the amplitude of the peaks of the Q or S wave and the peak of the T wave of the heartbeat pattern indicating the features of the subject. Accordingly, the processing of personal identification using the peak of the Q or S wave and the peak of the T wave of the heartbeat pattern stabilizes and the accuracy improves.

Common-mode signal generation circuit 13 further includes amplitude determination unit 40b. Amplitude determination unit 40b determines the amplitude of the common-mode signal to be generated based on the amplitude of the peaks of the ECG waveform. Signal generation unit 41 generates, as a common-mode signal, a signal with the amplitude determined by amplitude determination unit 40b.

This configuration allows application of the common-mode signal with the amplitude determined based on the amplitude of the peaks of the ECG waveform to one of electrodes 11. This increases an insufficient amplitude of the peaks of the ECG waveform. Accordingly, the processing of personal identification using the heartbeat pattern indicated by the ECG signal stabilizes and the accuracy improves.

Electrodes 11 attached to the living body include measurement electrode 11a and reference electrode 11b. Signal processing circuit 12 includes differential amplifier 32 and A/D converter 34. Differential amplifier 32 amplifies the difference between the signal detected by measurement electrode 11a and the signal detected by reference electrode 11b. A/D converter 34 converts the signal output from differential amplifier 32 into a digital signal. Common-mode signal generation circuit 13 applies a common-mode signal to reference electrode 11b using the digital signal output from A/D converter 34.

This configuration allows application of the common-mode signal generated based on the signal indicating the difference between the signal detected by measurement electrode 11a and the signal detected by reference electrode 11b to reference electrode 11b. Common-mode noise superimposed on each the signal is removed and stable ECG signals with less disturbance noise are generated.

Personal identification system 100 according to this embodiment includes ECG signal processor 10 described above, storage unit 23, and identification unit 22. Storage unit 23 stores, as register information, the features of the ECG waveform indicated by the ECG signals output from signal processing circuit 12 included in ECG signal processor 10 in association with the plurality of users. Identification unit 22 compares the features of the ECG waveform of the subject indicated by the ECG signals output from signal processing circuit 12 included in ECG signal processor 10 to the register information stored in storage unit 23. The identification unit identifies the subject among the plurality of users.

This allows personal identification using the ECG signals with the emphasized peaks of the heartbeat pattern. Accordingly, personal identification is performed stably and accurately even at a high contact impedance between electrodes 11 and the living body.

The ECG signal processing method according to this embodiment includes S10 of obtaining signals and S20 of generating a common-mode signal. In S10, the ECG signals are obtained which have been detected by electrodes 11 (i.e., measurement electrode 11a and reference electrode 11b) attached to the living body. In S20, the common-mode signal for increasing the amplitude of the peaks of the ECG waveform indicated by the ECG signals obtained in S10 of obtaining signals, and the generated common-mode signal is applied to reference electrode 11b.

This configuration allows application of the common-mode signal for increasing the amplitude of the peaks of the ECG waveform to one of electrodes 11 and emphasis of the peaks of the heartbeat pattern indicated by the ECG signals. Accordingly, stable personal identification is possible even in the presence of disturbance noise. That is, an ECG signal processing method is achieved which allows stable measurement of ECG signals even at a high contact impedance between electrodes 11 and a living body.

In the present invention, the steps included in the ECG signal processing method may be implemented as programs executed by a computer. Alternatively, the steps included in the personal identification method performed by information processor 20 may be implemented as programs executed by a computer. The steps may be implemented by a computer-readable storage medium, such as a CD-ROM, storing such programs.

Now, an ECG signal processor according to a variation of the embodiment will be described.

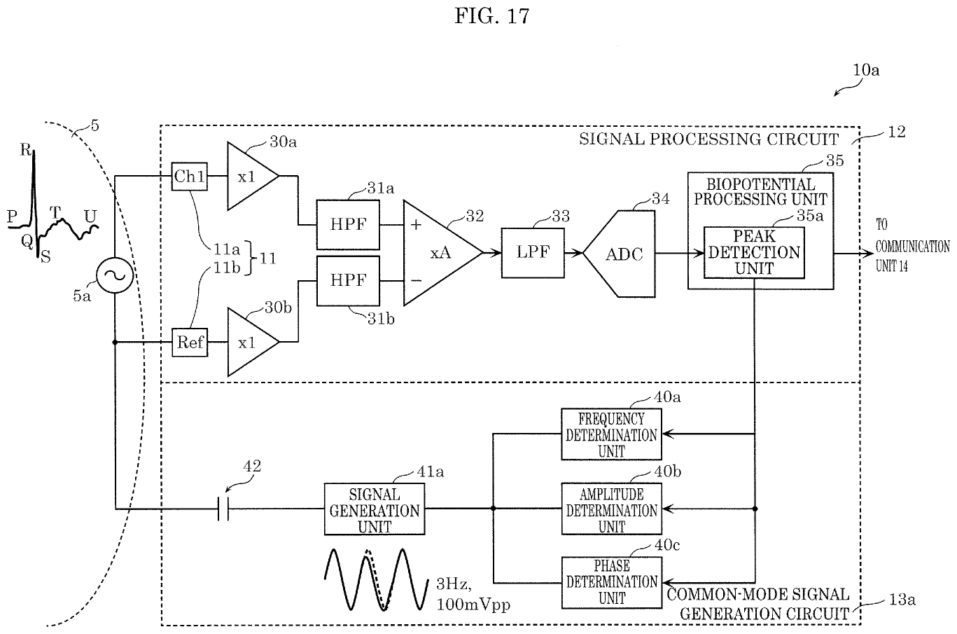

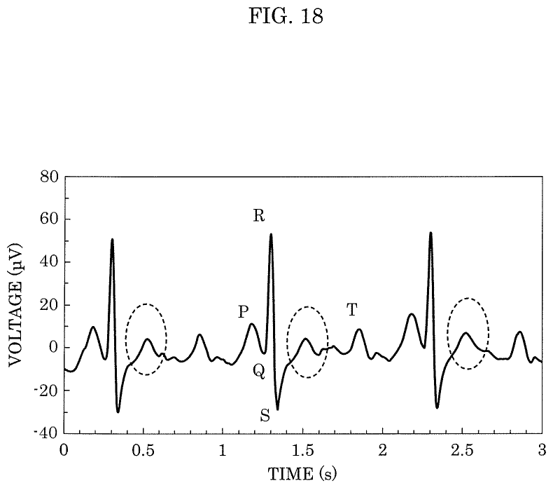

FIG. 17 is a block diagram illustrating a configuration of ECG signal processor 10a according to the variation of the embodiment. This ECG signal processor 10a corresponds to ECG signal processor 10 according to the embodiment described above including the following differences. Common-mode signal generation circuit 13 is replaced with common-mode signal generation circuit 13a that additionally includes phase determination unit 40c and includes signal generation unit 41a in place of signal generation unit 41.

Phase determination unit 40c generates a control signal for temporarily shifting the phase or temporarily reducing the amplitude of the common-mode signal to be generated. Specifically, phase determination unit 40c generates a common-mode signal with an example waveform shown in FIG. 17 to prevent or reduce erroneous detection of the T wave using the information on the peaks detected by peak detection unit 35a. In this variation, the phase determination unit generates, as a common-mode signal, the waveform with 1 Hz and three peaks repeated at 100 mVpp including a center peak with a smaller amplitude.

Signal generation unit 41a generates, as a common-mode signal, the signal including a part with a temporarily shifted phase or a temporarily reduced amplitude based on the control signal generated by phase determination unit 40c. Specifically, signal generation unit 41a generates a common-mode signal with the frequency determined by frequency determination unit 40a, the amplitude determined by amplitude determination unit 40b, and a part with the temporarily shifted phase or the temporarily reduced amplitude determined by phase determination unit 40c. That is, the signal generation unit generates such a sample data column. A built-in D/A converter converts the generated sample data column into an analog signal, and causes the analog signal to pass through a built-in low-pass filter.

Note that the digital signal processing in phase determination unit 40c and signal generation unit 41a may be implemented by hardware using an exclusive logic circuit or by software using programs. As software, the digital signal processing may be implemented by, for example, a microcomputer including a non-volatile memory, such as a ROM, storing programs; a RAM temporarily storing information; a processor executing programs; or input/output ports to be connected to peripherals.

FIG. 18 illustrates an example waveform (i.e., a waveform for registration and identification) of an ECG signal (hereinafter referred to as “registered data B”') on which a common-mode signal is superimposed by ECG signal processor 10a according to the variation. As can be seen from the comparison with the example waveform of registered data B described in the embodiment above and shown in FIG. 14, the unnecessary peak (i.e., the dashed frame in FIG. 18) between the S wave and the T wave is low. This increases the matching rates of the personal identification.

As described above, in ECG signal processor 10a according to this variation, common-mode signal generation circuit 13a includes phase determination unit 40c that generates a control signal for temporarily shifting the phase or temporarily reducing the amplitude of the common-mode signal to be generated. Signal generation unit 41a generates, as a common-mode signal, a signal including a part with a temporarily shifted phase or a temporarily reduced amplitude based on the control signal generated by phase determination unit 40c.

This configuration allows application of the common-mode signal including the part with the temporarily shifted phase or temporarily reduced amplitude to one of electrodes 11. This increases the amplitude of only the peak characteristic of the heartbeat pattern indicated by the ECG signals. Accordingly, processing of personal identification using the heartbeat pattern indicated by the ECG signals stabilizes and the accuracy improves.

The ECG signal processor, the personal identification system, and the ECG signal processing method according to the present invention have been described above based on the embodiments and variation. The present invention is however not limited to the embodiments and variation. The present invention may include other embodiments, such as those obtained by variously modifying the embodiments and variation as conceived by those skilled in the art or those achieved by freely combining the constituent elements in the embodiments and variation without departing from the scope and spirit of the present invention.

For example, while biopotential processing unit 35 is included in ECG signal processor 10 in the embodiments and variation described above, the configuration is not limited thereto. Instead or in addition, a/the biopotential processing unit may be included in information processor 20. If biopotential processing unit 35 is included in information processor 20, information on the peaks generated by peak detection unit 35a of biopotential processing unit 35 is utilized for generating signatures in identification unit 22.

If biopotential processing unit 35 is included in information processor 20, frequency determination unit 40a, amplitude determination unit 40b, and phase determination unit 40c of ECG signal processor 10 may also be included in information processor 20. In this case, the frequency, the amplitude, and the control signal determined by frequency determination unit 40a, amplitude determination unit 40b, and phase determination unit 40c, respectively, are sent via communication unit 21 of information processor 20 and communication unit 14 of ECG signal processor 10 to signal generation units 41 and 41a to ECG signal processor 10 to be utilized to generate as a common-mode signal.

Including both of frequency determination unit 40a and amplitude determination unit 40b in the embodiment described above, ECG signal processor 10 may include only one of the units. In this case, signal generation unit 41 generates a common-mode signal based on information obtained from frequency determination unit 40a and amplitude determination unit 40b.

Similarly, including all of frequency determination unit 40a, amplitude determination unit 40b, and phase determination unit 40c, ECG signal processor 10a may include at least one of the units. In this case, signal generation unit 41a generates a common-mode signal based on information obtained from at least one of frequency determination unit 40a, amplitude determination unit 40b, and phase determination unit 40c.

ECG signal processor 10a according to the embodiment described above may constitute a personal identification system, together with information processor 20 and display unit 25 according to the embodiment described above. This allows application of a common-mode signal including a part with a temporarily shifted phase or a temporarily reduced amplitude to one of electrodes 11, thereby increasing the amplitude of the peaks specific for the heartbeat pattern indicated by the ECG signals. Accordingly, processing of personal identification using the heartbeat pattern indicated by the ECG signals stabilizes and the accuracy improves.

In the embodiments and variation described above, ECG signal processors 10 and 10a process the signal detected by the measurement electrode using the potential detected by reference electrode 11b as a reference. The configuration is not limited thereto. The processors may process the signals detected by a plurality of measurement electrodes using the potential detected by the reference electrode as a reference. If multi-channel signals are processed by the ECG signal processors, a plurality of ECG waveforms obtained from the multi-channel signals may be, for example, averaged to be used for personal identification. Alternatively, a reference electrode is not always required. Only the signals of the measurement electrode may be processed using the ground potential using a reference. In this case, the common-mode signal is applied to the measurement electrode.

### REFERENCE MARKS IN THE DRAWINGS

5 subject

10, 10a ECG signal processor

11 electrode

11a measurement electrode

11b reference electrode

12 signal processing circuit

13, 13a common-mode signal generation circuit

22 identification unit

23 storage unit

32 differential amplifier

34 A/D converter

40a frequency determination unit

40b amplitude determination unit

40c phase determination unit

41, 41a signal generation unit

100 personal identification system

1. An electrocardiogram (ECG) signal processor, comprising:
a signal processing circuit configured to amplify an ECG signal detected by an electrode attached to a living body and output the ECG signal amplified; and
a common-mode signal generation circuit configured to generate a common-mode signal for increasing an amplitude of a peak of an ECG waveform indicated by the ECG signal using the ECG signal amplified by the signal processing circuit and apply the common-mode signal generated to the electrode.

2. The ECG signal processor according to claim 1, wherein
the common-mode signal generation circuit includes:
a frequency determination unit configured to determine a frequency corresponding to a time lag between a peak of a P wave and a peak of an R wave of the ECG waveform; and
a signal generation unit configured to generate, as the common-mode signal, a signal with the frequency determined by the frequency determination unit.

3. The ECG signal processor according to claim 1, wherein
the common-mode signal generation circuit includes:
a frequency determination unit configured to determine a frequency corresponding to a time lag between a peak of a Q wave or an S wave and a peak of a T wave of the ECG waveform; and
a signal generation unit configured to generate, as the common-mode signal, a signal with the frequency determined by the frequency determination unit.

4. The ECG signal processor according to claim 2, wherein
the common-mode signal generation circuit further includes:
an amplitude determination unit configured to determine an amplitude of the common-mode signal to be generated based on the amplitude of the peaks of the ECG waveform, and
the signal generation unit generates, as the common-mode signal, a signal with the amplitude determined by the amplitude determination unit.

5. The ECG signal processor according to claim 2, wherein
the common-mode signal generation circuit further includes:
a phase determination unit configured to generate a control signal for temporarily shifting a phase or temporarily reducing an amplitude of the common-mode signal to be generated, and
the signal generation unit generates, as the common-mode signal, a signal including a part with the phase temporarily shifted or the amplitude temporarily reduced based on the control signal generated by the phase determination unit.

6. The ECG signal processor according to claim 1, wherein
the electrode attached to the living body includes a measurement electrode and a reference electrode,
the signal processing circuit includes:
a differential amplifier configured to amplify a difference between a signal detected by the measurement electrode and a signal detected by the reference electrode; and
an A/D converter configured to converts a signal output from the differential amplifier into a digital signal, and
the common-mode signal generation circuit applies the common-mode signal to the reference electrode using the digital signal output from the A/D converter.

7. A personal identification system, comprising:
the ECG signal processor according to claim 1;
a storage unit configured to store, as register information, features of ECG waveforms indicated by ECG signals output from the signal processing circuit included in the ECG signal processor in association with a plurality of users; and
an identification unit configured to compare a feature of the ECG waveform of a subject indicated by the ECG signal output from the signal processing circuit included in the ECG signal processor with the register information stored in the storage unit to identify the subject among the plurality of users.

8. An electrocardiogram (ECG) signal processing method, comprising:
obtaining an ECG signal detected by an electrode attached to a living body; and
generating a common-mode signal for increasing an amplitude of a peak of an ECG waveform indicated by the ECG signal obtained in the obtaining of the ECG signal and applying the common-mode signal generated to the electrode.

9. A non-transitory computer-readable recording medium having recorded thereon a program causing a computer to execute the ECG signal processing method according to claim 8.
